(19) United States
(12) Patent Application Publication
(10) Pub. No.: US 2020/0359842 A1
(43) Pub. Date: Nov. 19, 2020

(54) SMART BLENDER AND INFORMATION PROVISION SYSTEM USING THE SAME

(71) Applicant: Sang wook KOH, Uijeongbu-si (KR)

(72) Inventor: Sang wook KOH, Uijeongbu-si (KR)

(21) Appl. No.: 16/844,024

(22) Filed: Apr. 9, 2020

(30) Foreign Application Priority Data
May 14, 2019 (KR) 10-2019-0056582

(51) Int. Cl. A47J 43/07; G16H 20/60; H04W 12/06; G06F 16/23; G05B 13/02; A47J 43/046; A47J 43/08; B01F 15/00

(52) U.S. Cl. CPC A47J 43/0716; G16H 20/60; H04W 12/06; G06F 16/2379; G05B 13/028; A47J 43/046; B01F 2215/0026; B01F 15/00194; B01F 15/00207; B01F 15/00311; A47J 2043/0733; B01F 2215/0014; A47J 43/085

(57) ABSTRACT

Disclosed herein are a smart blender and an information provision system using the same. The smart blender includes: a body part configured such that a drive motor is provided therein; a container part configured such that food ingredients are introduced thereinto; a grip part formed on the outer circumferential surface of the container part; a cover part detachably coupled to the top of the container part; a weight measurement portion configured to measure the weight of the food ingredients introduced into the container part; an information input/output portion configured to display the measured result of the weight measurement portion, and to receive information about the food ingredients and information about the number of persons from a user; and a control unit configured to determine the state of the food ingredients and control the operation of the blender according to the measured result of the weight measurement portion.

### CROSS-REFERENCE TO RELATED APPLICATION

This application claims the benefit of Korean Patent Application No. 10-2019-0056582 filed on May 14, 2019, which is hereby incorporated by reference herein in its entirety.

### BACKGROUND

### 1. Technical Field

Embodiments of the present invention relate to a smart blender and an information provision system using the same.

### 2. Description of the Related Art

Generally, a blender is beneficially used to provide juice, made by finely crushing a main ingredient such as a vegetable, a fruit, or the like and then mixing the crushed main ingredient with a minor ingredient such as milk, yogurt, and/or another additive, in a home, restaurant, or office.

The blender is configured to include a blender body configured to contain a drive motor therein, a blender cup configured to have an accommodation portion that accommodates a predetermined fruit, a vegetable, or the like, and blades configured to be disposed inside the blender cup, to be driven by a drive motor, and to finely crush an item (a fruit, or a vegetable).

The completeness, i.e., taste, of juice made using such a blender depends on whether or not a user has mixed a vegetable or fruit and milk, yogurt, or another additive at an appropriate ratio in accordance with the characteristics of the ingredients and the taste of an eater.

Accordingly, even when the same blender is used, the taste of juice varies depending on a user unless the ratio at which a fruit or vegetable and milk or another additive are mixed together is constant.

However, in the conventional juice provision method using a blender, ingredients are weighed and mixed together according to the experience or guess of a user. Accordingly, an unskilled person inevitably suffers from a difference in the mixing ratio. Therefore, the conventional juice provision method is problematic in that it is challenging to provide juice giving the same taste.

### RELATED ART DOCUMENTS

### Patent Documents

Patent document 1: Korean Patent No. 10-1071536 (issued on Oct. 14, 2011)

Patent document 2: Korean Patent Application Publication No. 10-2009-0124704 (published on Dec. 3, 2009)

### SUMMARY

Embodiments of the present invention provide a smart blender and information provision system using the same that accurately measure the weights of the ingredients of a recipe for a mixture with respect to a mixed edible food, calculate calories per unit gram based on the measured weights, and provide information optimized for a user, thereby satisfying the preference of the user and also improving user convenience.

According to an aspect of the present invention, there is provided a smart blender including: a body part configured such that a drive motor is provided therein; a container part configured such that food ingredients are introduced thereinto, and detachably coupled to the top of the body part; a grip part formed on the outer circumferential surface of the container part; a cover part detachably coupled to the top of the container part; a weight measurement portion disposed in the upper portion of the body part, and configured to measure the weight of the food ingredients introduced into the container part; an information input/output portion configured to display the measured result of the weight measurement portion, and to receive information about the food ingredients and information about the number of persons from a user; and a control unit configured to determine the state of the food ingredients and control the operation of the blender according to the measured result of the weight measurement portion; wherein the control unit includes a memory unit configured to store information about the weight of each of the food ingredients, information about a standard recipe using the food ingredient and information about an existing recipe using the food ingredient, information about amounts relative to the number of persons for the information about a standard recipe and the information about an existing recipe, and calorie information about each piece of information, and performs control to extract information about each of the food ingredients and information about a recommended recipe using the food ingredient and display the extracted information via the information input/output portion through the execution of a previously installed recipe recommendation program; wherein the information input/output portion includes: a main ingredient weight information input/output portion configured to indicate the weight of a main ingredient actually measured by the weight measurement portion; a mixing weight information input/output portion configured to display an appropriate mixing weight of a minor ingredient calculated based on an appropriate weight mixing ratio calculation formula for the minor ingredient of a recipe recommended based on the actually measured main ingredient; a minor ingredient weight information input/output portion configured to display the actually measured weight of the minor ingredient; and a remainder information input/output portion configured to display data related to a selected recommended recipe; wherein the appropriate weight mixing ratio is adjusted by the operation of the control unit in a stepwise manner, and a new weight mixing ratio calculation formula calculated by applying the actual weight mixing ratio between the main ingredient (a fruit or vegetable) and the minor ingredient (an auxiliary ingredient, such as milk, yogurt, or a separate additive, corresponding to the main ingredient) to an existing weight mixing ratio calculation formula is additionally stored, and the stored new weight mixing ratio calculation formula is used for the measurement of other ingredient mixing weights that is performed in the future; and wherein the control unit is connected to individual components and provided with a plurality of setting buttons configured to store data or input various setting items, and the setting buttons include various buttons, including: an on/off supply button configured to control the supply and cutoff of power; a zero-point setting button configured to reset a state of the weight measurement portion before measurement of ingredients; a calculation button configured to calculate weights of minor ingredients corresponding to an actually measured weight of a main ingredient; a standard mixing ratio setting/mixing ratio adjusting button configured to keep a weight mixing ratio of the ingredients equal to a basic weight mixing ratio or to adjust or select a mixing weight of any one of the ingredients in a stepwise manner; an erasing button configured to erase immediately previous states so that measurement of ingredients are newly started; a memory button configured to calculate a new calculation formula by applying an actual weight mixing ratio between a current main ingredient and a current minor ingredient to an existing weight mixing ratio calculation formula, and to store the new calculation formula; a cancellation button configured to cancel all inputs; character entry buttons configured to enter numbers and letters for data management; an information extraction button configured to extract information about a recommended recipe using information about each food ingredient; and a separate data exchange button configured to exchange and update data via a computer.

The smart blender may further include a communication unit configured to transmit and receive data to and from an external terminal, and the communication unit may transmit and receive the information about the weight of each of the food ingredients, the information about a standard recipe using the food ingredient and the information about an existing recipe using the food ingredient, the information about amounts relative to a number of persons for the information about a standard recipe and the information about an existing recipe, the calorie information about each piece of information, and the information about a recommended recipe.

According to another aspect of the present invention, there is provided an information provision system including: the above-described smart blender; and a user terminal configured to be connected with the smart blender through the execution of a previously installed information provision app, to receive the information about food ingredients, the information about the number of persons, and the weight information regarding the information about food ingredients from the smart blender, and to extract information about one of the food ingredients and information about a recommended recipe using this food ingredient and to display the extracted information or transmit the extracted information to the smart blender.

The user terminal may include: a connection module configured to be connected with the smart blender over a wireless communication network through the execution of an information provision app; an authentication module configured to perform a login operation of a user for requesting a connection via the connection module; a database configured to store information for the operation of the system (the information provision app recipe information, calorie information, user information, and mixture intake history information); a database management module configured to update or access the data of the database in response to a control signal; a recommended information provision module configured to provide information about calories of food ingredients and a mixture in which the food ingredients are mixed together when a user who has logged in via the authentication module inputs information about these food ingredients, information about the number of persons and information about weights or these pieces of information are received from the smart blender, and to provide information about a recommended recipe based on the information of the database; and a system control module configured to control the connection module performing user connection and the authentication module performing authentication, and to control operations of processing and updating data required for overall operation of the system via the database management module.

The database (DB) may include: a per-food ingredient recipe DB configured to store an average recipe for each of various food ingredients; a food ingredient DB configured to store information about calories per unit weight (1 g) for each of the various food ingredients; a user information DB configured to store information for user authentication and information including health and taste for each user; and a per-user intake history DB configured to store information about a mixture intake history for each user as the user who has logged in updates information about food ingredients.

### BRIEF DESCRIPTION OF THE DRAWINGS

The above and other objects, features, and advantages of the present invention will be more clearly understood from the following detailed description taken in conjunction with the accompanying drawings, in which:

FIG. 1 is a view showing a smart blender according to an embodiment of the present invention;

FIG. 2 is a view showing an information provision system according to another embodiment of the present invention; and

FIG. 3 is a flowchart illustrating the operation of the smart blender and information provision system using the same according to the embodiments of the present invention.

### DETAILED DESCRIPTION

The terms used herein will be described in brief, and the present invention will be described in detail.

General terms that are currently widely used are selected as the terms used herein while considering the functions thereof in the present invention, but the meanings thereof may vary according to the intention or precedent of a person skilled in the art or the advent of new technologies. In addition, in a specific case, a term arbitrarily selected by the applicant may also be used, in which case the meaning thereof will be described in detail in the corresponding portion of the description of the present invention. Therefore, the terms used herein should be defined based on the meanings thereof and the context of the present invention, rather than simply the names of the terms.

Throughout the specification, when any portion is described as including any component, this is not intended to exclude another component but is intended to include another component, unless particularly described to the contrary. Furthermore, the term “unit,” “module,” or the like described herein refers to a unit for processing at least one function or operation, which may be implemented as hardware, software, or a combination of hardware and software.

In the following, embodiments of the present invention will be described in detail with reference to the accompanying drawings so that those skilled in the art to which the present invention pertains may easily practice the present invention. However, the present invention may be implemented in various different forms and is not limited to the embodiments described herein. Furthermore, in the drawings, in order to clearly describe the present invention, parts irrelevant to the description of the present invention are omitted, and like reference symbols are assigned to similar components throughout the specification.

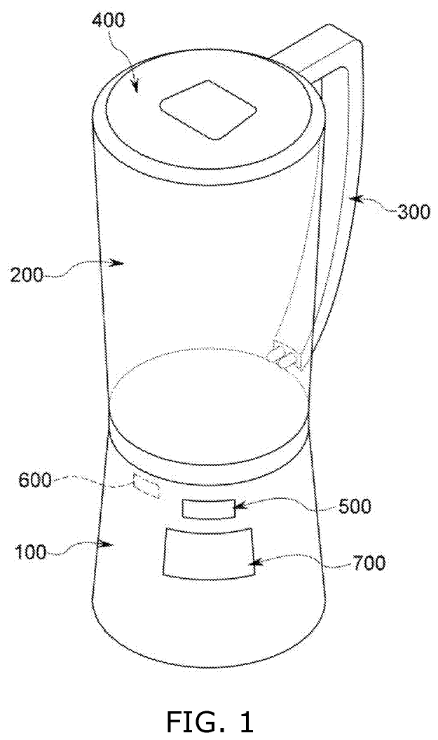

FIG. 1 is a view showing a smart blender according to an embodiment of the present invention.

As shown in FIG. 1, the smart blender according to the present embodiment is an appliance configured to contain and mix all edible foods such as raw foods, e.g., vegetables, fruits, etc., and includes a body part 100, a container part 200, a grip part 300, a cover part 400, an information input/output portion 500, a weight measurement portion 600, and a control unit 700.

The present invention is characterized in that a load cell configured to detect the weight of a food ingredient is installed on the inner bottom of the body part 10 of the smart blender.

The body part 100 constitutes the lower portion of the smart blender, and a drive unit including a drive motor is disposed in the body part 100. The overall configuration of the body part 100 is the same as or similar to that of a conventional blender.

The container part 200 is detachably coupled to the top of the body part 100. A food ingredient is introduced into the container part 200, and the food ingredient introduced into the container part 200 is crushed under the control of the control unit 700, to be described later, to form juice.

The grip part 300 is formed on the outer circumferential surface of the container part 200. A user may hold the container part 200 via the grip part 300.

The cover part 400 is detachably coupled to the top of the container part 200. Via the cover part 400, the food ingredient introduced into the container part 200 may be prevented from being removed from the smart blender to the outside while the food ingredient is being crushed, and a foreign material may be prevented from being introduced into the food ingredient.

The information input/output portion 500 displays the measured result of the weight measurement portion, and receives information about a food ingredient and information about the number of persons from a user. The information input/output portion 500 is configured in the form of a touchable display, such as an LCD, an LED, an OLED or the like, in order to display the measured result of the weight measurement portion 600, information about a food ingredient, information about the number of persons, and various types of menu items.

Furthermore, the information input/output portion 500 includes: a main ingredient weight information input/output portion configured to indicate the weight of a main ingredient actually measured by the weight measurement portion 600 so that the corresponding weight detected via the weight measurement portion 600 is displayed via the control unit 50; a mixing weight information input/output portion configured to display the appropriate mixing weight of the minor ingredient calculated based on an appropriate weight mixing ratio calculation formula for the minor ingredient of a recipe recommended based on the actually measured main ingredient; a minor ingredient weight information input/output portion configured to display the actually measured weight of the minor ingredient; and a remainder information input/output portion configured to display data related to the selected recommended recipe.

The weight measurement portion 600 is disposed on the upper portion of the body part 100, and measures the weight of food ingredients introduced into the container part 200.

Although not limited thereto, the weight measurement portion 600 may be composed of a pressure sensor.

The weight measurement portion 600 may be composed of a device, such as a load cell configured to detect weight by generating an electric signal in proportion to the deformation of an elastic body attributable to a load, or a weight-electromagnetic force equilibrating unit configured to detect weight by equilibrating the electrical detection of a displacement attributable to a load with electromagnetic force. Any other device may be used as long as it can measure the weight of food ingredients.

The control unit 700 identifies the states of food ingredients and also controls the operation of the blender based on the display results of the information input/output portion 500 and the measurement results of the weight measurement portion 600.

In other words, the control unit 700 may perform control so that the weight of the food ingredients is displayed on the information input/output portion 500 in response to a signal detected via the weight measurement portion 600. Furthermore, through the execution of a previously installed recipe recommendation program, the control unit 700 may perform control so that information about each food ingredient and information about a recommended recipe using the food ingredient are extracted from a memory unit (not shown) configured to store information about the weight of each food ingredient, information about a standard recipe using the food ingredient and information about an existing recipe using the food ingredient, information about amounts relative to the number of persons for the information about a standard recipe and the information about an existing recipe, and calorie information about each piece of information and are displayed via the information input/output portion 500.

Furthermore, the control unit 700 may be configured to display various mixing ratios, obtained by comparing the mixing ratio between the weights of the main ingredient and the minor ingredient displayed on the information input/output portion 500 with stored data, via the information input/output portion 500, or may be configured to output calculated appropriate mixing weights and/or the like via a digital display means such as a separate LCD or LED or the like.

Furthermore, the control unit 700 is configured to receive data on appropriate weight mixing ratio calculation formulae for various food ingredients in advance and to selectively use an appropriate weight mixing ratio calculation formula for a selected ingredient.

In this case, the appropriate weight mixing ratio may be adjusted by the operation of the control unit 700 in a stepwise manner. When necessary, a new weight mixing ratio calculation formula calculated by applying the actual weight mixing ratio between a main ingredient (a fruit or vegetable) and a minor ingredient (an auxiliary ingredient, such as milk, yogurt, or a separate additive, corresponding to the main ingredient) to an existing weight mixing ratio calculation formula may be additionally stored, and the stored new weight mixing ratio calculation formula may be used for the measurement of other ingredient mixing weights that is performed in the future.

Furthermore, the control unit 700 includes a memory unit (not shown) configured to store information about the weight of each food ingredient, information about a standard recipe using the food ingredient and information about an existing recipe using the food ingredient, information about amounts relative to the number of persons for the information about a standard recipe and the information about an existing recipe, and calorie information about each piece of information.

For example, the memory unit is disposed below the cover part 400 in order to provide information about the weight of each food ingredient crushed in response to a signal of the control unit 700, information about a standard recipe using the food ingredient and information about an existing recipe using the food ingredient, information about amounts relative to the number of persons for the information about a standard recipe and the information about an existing recipe, and calorie information about each piece of information.

In addition, the memory unit stores the weight data of the container part 200 before a food ingredient is introduced thereinto. In other words, as the weight data of the container part 200 is stored, the weight of a food ingredient currently introduced into the container part 200 is calculated by subtracting the initial weight of the previously stored container part 200 from the weight measured by the weight measurement portion 600.

Furthermore, the memory unit stores the drive speed data of the drive motor mounted in the smart blender and the drive time data of the drive motor based on the weight of food ingredients introduced into the container part 200. For example, the above data is stored in the form of a lookup table in such a manner that the drive time is 1 minute when the weight of the food ingredients introduced into the container part 200 is 1 kilogram, the drive time is 30 seconds when the weight of the food ingredients introduced into the container part 200 is 300 grams and so forth. Data not stored in the memory unit is calculated using an interpolation method.

Furthermore, the control unit 700 further includes a communication unit (not shown) configured to transmit and receive data to and from an external terminal.

In this case, the communication unit transmits and receives information about the weight of each food ingredient, information about a standard recipe using the food ingredient and information about an existing recipe using the food ingredient, information about amounts relative to the number of persons for the information about a standard recipe and the information about an existing recipe, calorie information about each piece of information, and information about a recommended recipe.

The communication unit converts a signal, stored in the memory unit, into a digital signal and transmits the digital signal to the information input/output portion 500. The information about the weight of each food ingredient, the information about a standard recipe using the food ingredient and the information about an existing recipe using the food ingredient, the information about amounts relative to the number of persons for the information about a standard recipe and the information about an existing recipe, the calorie information about each piece of information, and the information about a recommended recipe are displayed from the memory unit onto the information input/output portion 500 via the communication unit, a user can visually check the weights, calories, and recipe information of desired ingredients and mixtures using the desired ingredients, thereby improving reliability.

Furthermore, the control unit 700 is connected to individual components, and is provided with a plurality of setting buttons (not shown) configured to store data or input various setting items.

The setting buttons include various buttons, including: an on/off supply button configured to control the supply and cutoff of power; a zero-point setting button configured to reset the state of the weight measurement portion 600 before the measurement of ingredients; a calculation button configured to calculate the weights of minor ingredients corresponding to the actually measured weight of a main ingredient; a standard mixing ratio setting/mixing ratio adjusting button configured to keep the weight mixing ratio of the ingredients equal to a basic weight mixing ratio or to adjust or select the mixing weight of any one of the ingredients in a stepwise manner; an erasing button configured to erase immediately previous states so that the measurement of ingredients may be newly started; a memory button configured to calculate a new calculation formula by applying the actual weight mixing ratio between a current main ingredient and a current minor ingredient to an existing weight mixing ratio calculation formula, and to store the new calculation formula; a cancellation button configured to cancel all inputs; character entry buttons configured to enter numbers and letters for data management; an information extraction button configured to extract recommended recipe information using information about each food ingredient; and a separate data exchange button (a USB port) configured to exchange and update data via a computer.

Furthermore, the control unit 700 is provided with the separate data exchange button (a USB port) to exchange and update data via a computer, and thus only data may be downloaded or transmitted to a desired location over the Internet when necessary. Accordingly, in the case of juices or other mixtures having special purposes in the form of healthy foods for users, it may be possible to reduce the burdensomeness or inconvenience of purchasing, carrying and/or moving a separate blender.

Meanwhile, the control unit 700 includes: a Bluetooth module configured to, in preparation for a case where a plurality of users wants to access and use, always transmit a Bluetooth signal, be wirelessly connected to a user terminal located within a first distance range from the smart blender, receive the key value of a first user from an external terminal, and output a control signal to the external terminal when the authentication of the received key value of the first user is completed; and a human body sensor configured to detect a user approaching within a second distance range shorter than the first distance range from the smart blender and to output a user detection signal to the Bluetooth module.

In this case, the user terminal includes a user app configured to wirelessly connect the user terminal to the Bluetooth module within the first distance range and to provide a previously stored user key value to the Bluetooth module when the user terminal is located within the third distance range between the first and second distance ranges.

Furthermore, the Bluetooth module performs the process of authenticating the user key value received from the user app when receiving the user detection signal, measures the time interval between the time at which the user key value is received from the user app and the time at which the user detection signal is received, and does not perform the authentication process when the measured time interval is shorter than a preset reference time interval, so that, regardless of the surrounding environment, the season, the weather, and the mobile phone manufacturer and model, the distance between the user and the smart blender for the use of smart blender use, the time at which the smart blender needs to be prepared to start running, the history of the use of the blender of each user, and/or unique recipes may be consistently guaranteed and secured and there may be solved an error in the preparation of the operation of a smart blender that may be caused by two or more users.

As described above, the smart blender according to the present invention may accurately measure the weights of the ingredients of a recipe for a mixture with respect to a mixed edible food, may calculate calories per unit gram based on the measured weights, and may provide information optimized for a user, thereby satisfying the preference of the user and improving user convenience. Furthermore, the smart blender according to the present invention may calculate and provide the taste and calories of a unique recipe, so that both taste and nutrition may be managed and a mixture based on the unique recipe may be continuously made and eaten.

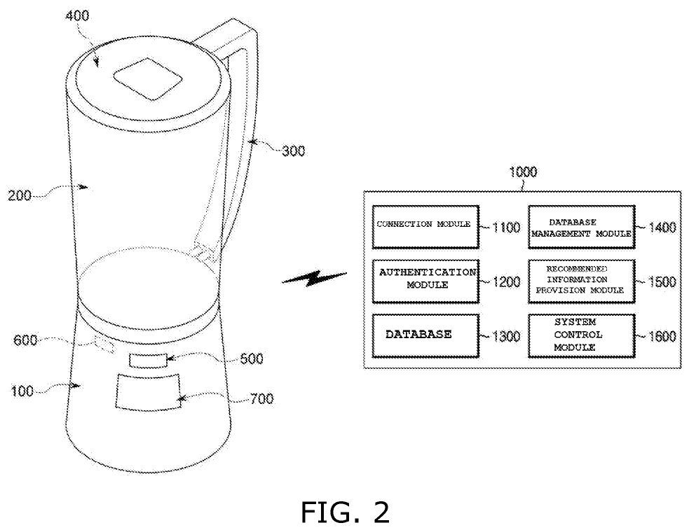

FIG. 2 is a view showing an information provision system according to another embodiment of the present invention.

As shown in FIG. 2, the information provision system according to the present embodiment includes the smart blender shown in FIG. 1, and a user terminal 1000 configured to be connected with the smart blender through the execution of a previously installed information provision app, to receive information about food ingredients, information about the number of persons, and weight information regarding the information about food ingredients from the smart blender, and to extract information about each of the food ingredients and information about a recommended recipe using the food ingredient and to display the extracted information or transmit the extracted information to the smart blender.

The user terminal 1000 may be implemented as a computer or wireless communication device that is connectable to the smart blender over a network through the execution of an app. In this case, the computer may include, for example, a notebook, a desktop, a laptop, etc. equipped with a navigation system and a web browser. In this case, the at least one user terminal 100 may be implemented as a terminal that is connectable to a remote server or terminal over a network. The wireless communication device is a device that guarantees portability and mobility, and may include all types of handheld wireless communication devices, such as a navigation system, Personal Communication System (PCS), Global System for Mobile communications (GSM), Personal Digital Cellular (PDC), Personal Handyphone System (PHS), Personal Digital Assistant (PDA), International Mobile Telecommunication (IMT)-2000, Code Division Multiple Access (CDMA)-2000, W-Code Division Multiple Access (W-CDMA), and Wireless Broadband Internet (Wibro) terminals, a smartphone, a smart pad, a tablet PC, and the like.

More specifically, the user terminal 1000 includes: a connection module 1100 configured to be connected with the smart blender over a wireless communication network through the execution of an information provision app; an authentication module 1200 configured to perform the login operation of a user for requesting a connection via the connection module 1100; a database 1300 configured to store information for the operation of the system (an information provision app, recipe information, calorie information, user information, and mixture intake history); a database management module 1400 configured to update or access the data of the database 1300 in response to a control signal; a recommended information provision module 1500 configured to provide information about the calories of food ingredients and a mixture in which the food ingredients are mixed together when a user who has logged in via the authentication module 1200 inputs information about food ingredients, information about the number of persons and information about weights or they are received from the smart blender, and to provide information about a recommended recipe based on the information of the database 1300; and a system control module 1600 configured to control the connection module 1100 configured to perform user connection and the authentication module 1200 configured to perform authentication, and to control the operations of processing and updating data required for the overall operation of the system via the database management module 1400.

In this case, although not shown, the database (DB) 1300 may be configured to include: a per-food ingredient recipe DB configured to store an average recipe for each of various food ingredients; a food ingredient DB configured to store information about calories per unit weight (1 g) for each of various food ingredients; a user information DB configured to store information for user authentication and information including health and taste for each user; and a per-user intake history DB configured to store information about a mixture intake history for each user as the user who has logged in updates information about food ingredients.

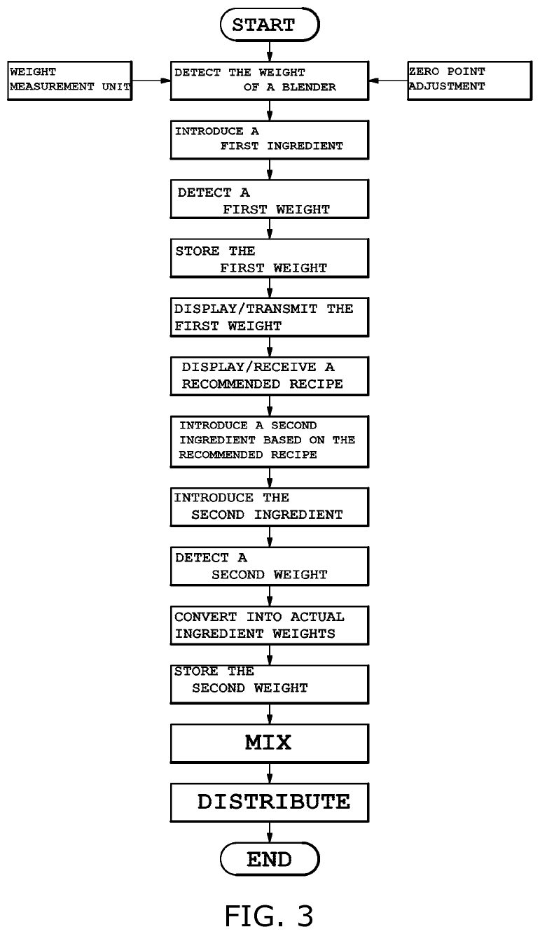

FIG. 3 is a flowchart illustrating the operation of the smart blender and information provision system using the same according to the embodiments of the present invention.

As shown in FIG. 3, in the information provision system using the smart blender according to the present embodiment, the container part 200 is fitted into the top of the body part 100 of the smart blender, and the weight measurement portion 600 detects the weight of the container part 200 and allows the weight to be displayed on the information input/output portion 500. In this case, the weight of the container part 200 detected via the weight measurement portion 600 is the container part 200's own weight in the state in which items, i.e., ingredients, have not been introduced into the container part 200, and thus the weight should be excluded.

Accordingly, in order to delete the detected container part 200's own weight, the setting button of the control unit 700 is operated to reset the scale of the weight measurement portion 600 to the state of zero “0.”

When the state of the weight measurement portion 600 is reset, a first ingredient, e.g., one main ingredient (a first ingredient in FIG. 3) selected from a fruit and a vegetable is introduced into the container part 200.

As the predetermined main ingredient is introduced into the container part 200, the weight measurement portion 600 detects the weight of the main ingredient, and the actually measured weight of the corresponding main ingredient is displayed in grams (kg, or g) via the information input/output portion 500, thereby notifying a user of the corresponding weight.

Accordingly, the user determines the amount of the main ingredient while comparing the weight of the main ingredient displayed via the information input/output unit 500, and then stops the introduction of the main ingredient when a corresponding amount of main ingredient is introduced.

The weight of the main ingredient displayed on the information input/output portion 500 as described above is set using the setting button of the control unit 700 such that the actually measured weight value is fixed to the main ingredient information input/output portion 500.

Thereafter, according to a value set in the information input/output portion 500, the control unit 700 may extract information about a main ingredient and information about a recommended recipe using the main ingredient through the execution of a recipe recommendation program on which data on a corresponding weight value has been previously installed (or according to a value set in the information input/output portion 500, the control unit 700 may compare data on a corresponding weight value and may calculate the weight of a minor ingredient that forms a preferred mixture for a weight actually measured by a weight mixing ratio calculation formula) and may display the information via the information input/output portion 500, or may be connected with the user terminal 1000 through the execution of a previously installed information provision app, may receive information about a first ingredient and information about a recommended recipe using the first ingredient from the user terminal 1000 and may display the information.

In this case, the control unit 700 may perform control to extract information about each food ingredient and information about a recommended recipe using the food ingredient and may display the extracted information via the information input/output portion 500 through the execution of a previously installed recipe recommendation program, or, in response to the selection of a user, may transfer information about the weight of each food ingredient, information about a standard recipe using the food ingredient and information about an existing recipe using the food ingredient, information about amounts relative to the number of persons for the information about a standard recipe and the information about an existing recipe, calorie information about each piece of information, and information about a recommended recipe to the app of the user terminal and may receive information about the calories of the food ingredient and a mixture including the food ingredient and information about a recommended recipe based on the calorie information from the user terminal.

Furthermore, the information input/output portion 500 displays the measured result of the weight measurement portion 600, and receives information about food ingredients and information about the number of persons from the user.

In this case, the user processes a minor ingredient (a second ingredient in FIG. 3) included in the recommended recipe information in the same manner as the main ingredient is introduced, detected, and stored, finally forming a mixture through mixing.

Furthermore, it will be apparent that the minor ingredient includes at least one ingredient.

Meanwhile, the app installed on the smart blender or user terminal 1000 in the present invention generates a new or novel recipe, i.e., a recommended recipe, corresponding to information about each food ingredient from the recipe data stored in each memory unit and database by using a deep learning technique. In this case, the recipe data stored in each memory unit and database becomes models of learning to be learned through a deep learning technique.

Furthermore, the recommended recipe may be designed to select and determine whether to modify a new recipe according to the preference of the user.

Embodiments of the present invention provide the smart blender and information provision system using the same that accurately measure the weights of the ingredients of a recipe for a mixture with respect to a mixed edible food, calculate calories per unit gram based on the measured weights, and provide information optimized for a user, thereby satisfying the preference of the user and improving user convenience.

Furthermore, embodiments of the present invention calculate and provide the taste and calories of a unique recipe, so that both taste and nutrition may be managed and a mixture based on the unique recipe may be continuously made and eaten.

The above-described embodiments are merely examples illustrating the smart blender and information provision system using the same according to the present invention. The present invention is not limited to these embodiments. It will be apparent to those having ordinary skill in the art to which the present invention pertains that various modifications and alterations may be made without departing from the spirit and scope of the attached claims.

What is claimed is:

1. A smart blender comprising:
a body part configured such that a drive motor is provided therein;
a container part configured such that food ingredients are introduced thereinto, and detachably coupled to a top of the body part;
a grip part formed on an outer circumferential surface of the container part;
a cover part detachably coupled to a top of the container part;
a weight measurement portion disposed in an upper portion of the body part, and configured to measure a weight of the food ingredients introduced into the container part;
an information input/output portion configured to display a measured result of the weight measurement portion, and to receive information about the food ingredients and information about a number of persons from a user; and
a control unit configured to determine a state of the food ingredients and control operation of the blender according to the measured result of the weight measurement portion;
wherein the control unit comprises:
a memory unit configured to store information about a weight of each of the food ingredients, information about a standard recipe using the food ingredient and information about an existing recipe using the food ingredient, information about amounts relative to the number of persons for the information about a standard recipe and the information about an existing recipe, and calorie information about each piece of information, and
performs control to extract information about each of the food ingredients and information about a recommended recipe using the food ingredient and display the extracted information via the information input/output portion through execution of a previously installed recipe recommendation program;
wherein the information input/output portion comprises: a main ingredient weight information input/output portion configured to indicate a weight of a main ingredient actually measured by the weight measurement portion; a mixing weight information input/output portion configured to display an appropriate mixing weight of a minor ingredient calculated based on an appropriate weight mixing ratio calculation formula for the minor ingredient of a recipe recommended based on the actually measured main ingredient; a minor ingredient weight information input/output portion configured to display an actually measured weight of the minor ingredient; and a remainder information input/output portion configured to display data related to a selected recommended recipe;
wherein the appropriate weight mixing ratio is adjusted by operation of the control unit in a stepwise manner, and a new weight mixing ratio calculation formula calculated by applying an actual weight mixing ratio between the main ingredient (a fruit or vegetable) and the minor ingredient (an auxiliary ingredient, such as milk, yogurt, or a separate additive, corresponding to the main ingredient) to an existing weight mixing ratio calculation formula is additionally stored, and the stored new weight mixing ratio calculation formula is used for measurement of other ingredient mixing weights that is performed in a future; and
wherein the control unit is connected to individual components and provided with a plurality of setting buttons configured to store data or input various setting items, and the setting buttons comprise various buttons, including: an on/off supply button configured to control supply and cutoff of power; a zero-point setting button configured to reset a state of the weight measurement portion before measurement of ingredients; a calculation button configured to calculate weights of minor ingredients corresponding to an actually measured weight of a main ingredient; a standard mixing ratio setting/mixing ratio adjusting button configured to keep a weight mixing ratio of the ingredients equal to a basic weight mixing ratio or to adjust or select a mixing weight of any one of the ingredients in a stepwise manner; an erasing button configured to erase immediately previous states so that measurement of ingredients are newly started; a memory button configured to calculate a new calculation formula by applying an actual weight mixing ratio between a current main ingredient and a current minor ingredient to an existing weight mixing ratio calculation formula, and to store the new calculation formula; a cancellation button configured to cancel all inputs; character entry buttons configured to enter numbers and letters for data management; an information extraction button configured to extract recommended recipe information using information about each food ingredient; and a separate data exchange button configured to exchange and update data via a computer.

2. The smart blender of claim 1, further comprising a communication unit configured to transmit and receive data to and from an external terminal;
wherein the communication unit transmits and receives the information about a weight of each of the food ingredients, the information about a standard recipe using the food ingredient and the information about an existing recipe using the food ingredient, the information about amounts relative to a number of persons for the information about a standard recipe and the information about an existing recipe, the calorie information about each piece of information, and the information about a recommended recipe.

3. An information provision system comprising:
the smart blender set forth in claim 1; and
a user terminal configured to be connected with the smart blender through execution of a previously installed information provision app, to receive the information about food ingredients, the information about the number of persons, and the weight information regarding the information about food ingredients from the smart blender, and to extract information about one of the food ingredients and information about a recommended recipe using this food ingredient and to display the extracted information or transmit the extracted information to the smart blender.

4. An information provision system comprising:
the smart blender set forth in claim 2; and
a user terminal configured to be connected with the smart blender through execution of a previously installed information provision app, to receive the information about food ingredients, the information about the number of persons, and the weight information regarding the information about food ingredients from the smart blender, and to extract information about one of the food ingredients and information about a recommended recipe using this food ingredient and to display the extracted information or transmit the extracted information to the smart blender.

5. The information provision system of claim 3, wherein the user terminal comprises:
a connection module configured to be connected with the smart blender over a wireless communication network through execution of an information provision app;
an authentication module configured to perform a login operation of a user for requesting a connection via the connection module;
a database configured to store the information provision app, recipe information, calorie information, user information, and mixture intake history information for operation of the system;
a database management module configured to update or access data of the database in response to a control signal;
a recommended information provision module configured to provide information about calories of food ingredients and a mixture in which the food ingredients are mixed together when a user who has logged in via the authentication module inputs information about these food ingredients, information about a number of persons and information about weights or these pieces of information are received from the smart blender, and to provide information about a recommended recipe based on the information of the database; and
a system control module configured to control the connection module performing user connection and the authentication module performing authentication, and to control operations of processing and updating data required for overall operation of the system via the database management module.

6. The information provision system of claim 4, wherein the database (DB) comprises:
a per-food ingredient recipe DB configured to store an average recipe for each of various food ingredients;
a food ingredient DB configured to store information about calories per unit weight (1 g) for each of the various food ingredients;
a user information DB configured to store information for user authentication and information including health and taste for each user; and
a per-user intake history DB configured to store information about a mixture intake history for each user as the user who has logged in updates information about food ingredients.
